(19) United States
(12) Patent Application Publication
(10) Pub. No.: US 2010/0197571 A1
(43) Pub. Date: Aug. 5, 2010

(54) EXTRUDATES WITH IMPROVED TASTE MASKING

(75) Inventors: Venkata-Rangarao Kanikanti, Leverkusen (DE); Hans-Juergen Hamann, Dormagen (DE); Peter Kleinebudde, Duesseldorf (DE); Andrea Michalk, Wiesbaden (DE); Claudia Reitz, Basel (CH)

Correspondence Address: BAYER HEALTHCARE LLC, P.O.BOX 390, SHAWNEE MISSION, KS, 66201, US

(73) Assignee: BAYER ANIMAL HEALTH GMBH, LEVERKUSEN (DE)

(21) Appl. No.: 12/602,002

(22) PCT Filed: May 28, 2008
(86) PCT No.: PCT/EP2008/004218

§ 371 Date: Feb. 11, 2010

(30) Foreign Application Priority Data
Jun. 8, 2007 (DE) 102007026550.8

(51) Int. Cl. A61K 38/12; A61K 31/66; A61K 31/4985

(52) U.S. Cl. 514/11; 514/114; 514/250

(57) ABSTRACT

The invention relates to extrudates comprising one or more pharmaceutically active substances, where the extrudates have a strand diameter of 0.5 mm or less, and to the use of these extrudates for the manufacture of medicaments.

The invention relates to extrudates comprising one or more pharmaceutically active substances, where the extrudates have a strand diameter of 0.5 mm or less, and to the use of these extrudates for the manufacture of medicaments.

Controlled release of medicinal substances has the advantage for consumers of being able to conceal the unpleasant taste of active ingredients. This increases the readiness to take the respective pharmaceutical form, as is important for optimal therapy. There are in this connection various possibilities for concealing taste in pharmaceutical technology. An overview of very many methods, together with cross references to appropriate literature sources is given by Roy [Roy, 1994] or Sohi [Sohi et al., 2004].

The simplest way of concealing taste is to add flavourings, but the concealing of very bitter and very readily water-soluble substances may be problematic. The procedure for finding the correct additions is described by Bienz [Bienz, 1996].

Taste masking by processing the active ingredient (hexahydropyrazine derivatives) with a hydrophobic carrier to give granules has also been described (WO 98/03157).

Another, frequently described possibility is to employ coatings on pharmaceutical forms. Besides protection from environmental influences, it is possible by means of a coating to control the release of the active ingredient from the pharmaceutical form in various ways, inter alia resulting in a concealing of taste. Materials used for this purpose may differ in origin and structure, for example Eudragit E [Cerea et al., 2004, Lovrecich et al., 1996, Ohta and Buckton, 2004, Petereit and Weisbrod, 1999], shellac [Pearnchob et al., 2003b, Pearnchob et al., 2003a] or cellulose derivatives [Al-Omran et al., 2002, Li et al., 2002, Shirai et al., 1993]. The disadvantage of using Eudragit E is, however, that the taste masking derives from an ionic interaction between the cationic excipient and anionic active ingredients. The use of shellac is likewise not advantageous because it is a natural polymer whose composition may vary. Apart from this, coatings involve further labour in the manufacture, causing expenditure of time and money. However, WO 2002/058669 describes a solid dispersion of quinolone- or naphthyridonecarboxylic acids in an insoluble matrix, and a particular possibility is a shellac matrix.

The use of ion exchange resins or inclusion complexes may likewise be suitable for taste masking. However, ion exchange resins lack broad applicability for many medicinal substances, because ionic properties must be present [Chun and Choi, 2004, Lu et al., 1991, Prompruk et al., 2005]. Inclusion complexes have the disadvantage that only low loading with active ingredient is possible [Sohi et al., 2004].

Fatty bases are likewise used in the manufacture of taste-concealing pharmaceutical forms. Investigations on monolithic pharmaceutical forms based on hard fat (Witepsol, Witocan) [Suzuki et al., 2003, Suzuki et al., 2004], where lecithin and sweeteners are additionally employed to improve the taste, are known. The disadvantage in this case is that the fatty bases must be melted, in turn possibly leading to instabilities. The cast tablets with a diameter of 2 cm are too large to be able to draw conclusions about use in the animal feed sector on the basis of these data. In addition, comparisons between hard fat, glycerol distearate and stearic acid as lipophilic binders in cold extrusion [Breitkreutz et al., 2003] have been undertaken, and in this case it was necessary to employ Eudragit E as coating in order to conceal the taste. The extrusion of fats below their melting point to manufacture pharmaceutical bases has likewise been described [Reitz and Kleinebudde, 2007].

EP 855 183 A2 discloses taste-masked oral formulations with gyrase inhibitors of the quinolone type, which are manufactured by the active ingredient being mixed with higher fatty acids and, where appropriate, further additives, heated and, after cooling, granulated or powdered.

Pellets based on waxes have also been produced [Adeyeye and Price, 1991, Adeyeye and Price, 1994, Zhou et al., 1996, Zhou et al., 1998]. In this case it was found that release of the active ingredients depends on the melting point of the wax and its concentration in the pellet. Release became slower as the melting point and wax content increased.

A further possibility for concealing taste is described by Kin and Choi [Kim and Choi, 2004] who produced a fatty core of cocoa butter or hard fat and the active ingredient and provided it with a shell of sodium alginate or carrageenan. However, in this case, the fat is completely melted and the coating step in the production represents an additional operation.

In addition, Compritol® 888 ATO has been described as matrix-forming component [Mirghani et al., 2000]. They describe a manufacture of pellets consisting of molten Compritol®, active ingredient and a polysaccharide covering. The coating with the polysaccharide is once again an operation which ought to be dispensed with. Li [Li et al., 2006] by contrast described comparison of matrix tablets manufactured by compression in a rotary machine either from a powder mixture or from a powdered solid dispersion. The tablets from the powdered solid dispersion showed better taste masking. However, the Compritol® 888 ATO was completely melted to produce the solid dispersion. Barthelemy [Barthelemy et al., 1999] used Compritol® 888 ATO for coating theophylline pellets and granules. Once again, the fat was completely melted.

In addition, the use of phospholipids is a possibility for improving the taste. It has been found in this connection that phospholipids mask only a bitter taste but have no influence on other taste sensations [Katsuragi et al., 1997, Takagi et al., 2001]. On the one hand, therefore, there is no possibility of universal application here because only a bitter taste can be concealed and, on the other hand, it is known that addition of phospholipids influences the crystallinity of lipids, possibly leading to instabilities [Schubert, 2005].

A further study showed that the organization of a powder mixture can likewise contribute to taste masking [Barra et al., 1999]. The excipient particles (cellulose derivatives) must be smaller than the active ingredient particles in order to make the concealing possible, because the excipient particles are deposited on the active ingredient particles. The disadvantage in this case is an adequate size of active ingredient particles, precluding the use of micronized substances.

WO 2003/030653 relates to animal feed in which active ingredients are incorporated and which can be produced by extrusion.

WO 2003/072083 describes the melt extrusion of a mixture of a basic medicinal substance and of a (meth)acrylate polymer; the extrudates are subsequently comminuted to granules or a powder. Taste-sealing of the active ingredient is said to be achieved in the resulting product. WO 2004/066976 discloses a process for producing an oral pharmaceutical form with immediate disintegration by mixing an anionic active ingredient, methacrylate polymer and a medium- to long-chain fatty acid in the melt. After solidification, the product is ground and incorporated in a water-soluble matrix.

U.S. Pat. No. 6,171,615 B1 relates to a sustained-release formulation of theophylline in a semisolid matrix comprising polyglycolysed glycerides and a mixture of substances to improve the formation of crystal nuclei (“nucleation enhancers”). FR 2 753 904 relates to a medicament with controlled release which comprises the active ingredient in a lipid matrix which in turn includes a behenic ester and a hydrophobic diluent.

WO 2004/014346 relates to a palatable formulation with controlled release which is suitable for companion animals. The formulation comprises the active ingredient in a small-particle (“multiparticulate”) form which is suitable for controlled release, and an addition which improves palatability.

WO 2005/097064 relates to a medicament which comprises a large number of coated particles whose core comprises a matrix material and a water-swellable swelling agent.

It has now been found that extrudates are very suitable for manufacturing taste-masked preparations or preparations with concealed taste, where the strand diameter in particular has an unexpected importance. A skilled person normally expects increased release of the ingredients with smaller particles and thus a poorer concealing of taste. Usual pharmaceutically used extrudates are produced with a strand diameter of the order of about 1 mm. It has now been found that when the strand diameter is reduced there is likewise a reduction in the release of the ingredients, so that extrudates with a smaller strand diameter can be used to manufacture medicaments with concealed taste.

The invention therefore relates to:
extrudates comprising one or more pharmaceutically active substances, characterized in that the extrudate has a strand diameter of 0.5 mm or less.
the use of the aforementioned extrudates for the manufacture of medicaments.

The strand diameter of the extrudates of the invention does not exceed 0.5 mm and preferably does not exceed 0.3 mm. Extrudates with a diameter of from 0.2 mm onwards can normally be used. In the case of non-cylindrical extrudates, the maximum edge length or ellipse length does not exceed 0.5 mm and preferably does not exceed 0.3 mm.

The extrudates comprise a base which is suitable for extrusion and consists of a thermoformable material or a mixture of a plurality of thermoformable materials, and where appropriate further pharmaceutically acceptable excipients and additives.

The base consists of thermoformable materials such as polymers, for example polyacrylates or cellulose derivatives, lipids, for example acyl glycerides, surfactants, for example glycerol monostearate or sodium stearate, macrogols, for example polyethylene glycol 6000, sugars or sugar alcohols, for example mannitol or xylitol. A lipid base is preferably used. Examples suitable as lipid base are fatty bases, in particular glycerol esters, preferably esters with C₁₂-C₂₄ fatty acids. Glycerol esters which may be mentioned are glycerol diesters such as, for example, glycerol dibehenate, glycerol triesters such as, for example, glycerol trilaurate, glycerol myristate, glycerol tripalmitate or glycerol tristearate, mixtures of glycerol mono-, di- and triesters such as, for example, glycerol palmitostearate. Mention may also be made of triglycerides based on coconut fat, palm oil and/or palm kernel oil (such as, for example, the hard fats commercially available under the name Witocan®). Mono- or diglycerides of citric and/or lactic acid can also be employed.

Mention may furthermore be made of waxes, especially those having 30 to 60 carbon atoms, such as cetyl palmitate. Such lipids are commercially available for example under the names Precirol®, Compritol® and Dynasan®. Particularly preferred examples are glycerol dibehenate and glycerol trimyristate. The fatty bases are preferably in powder form. Many lipids are polymorphic and may in some circumstances form metastable forms when the temperature and pressure change. During storage, in some circumstances, transformations of the modifications may occur and more stable modifications form. According to descriptions in the literature [Reitz and Kleinebudde, 2007], glycerol trimyristate (Dynasan 114®) is comparatively stable towards such changes and is therefore particularly suitable as lipid base for medicaments.

The substances used in particular as fatty bases are often marketed as mixtures, e.g. of mono-, di- and/or triglycerides. Compared with these, preference is given to uniform fatty bases which consist essentially of only one component. Formulations produced with these excipients are distinguished by good storage stability.

The amount of base (of thermoformable materials) employed depends on the amount of the other ingredients of the extrudates. Normally, from 20 to 99% [m/m], preferably 25 to 80% [m/m], particularly preferably 30 to 70% [m/m], are employed.

The extrudates of the invention may where appropriate comprise one or more further excipients and additives. Suitable as such are: flow regulators, preferably colloidal silicon dioxide in a concentration of from 0.2% to 2% [m/m]; lubricants, preferably magnesium stearate or calcium dibehenate in a concentration of from 0.2% to 5% [m/m]; surfactants, preferably lecithin in a concentration of from 0.5% to 10% [m/m]. It is further possible to employ antioxidants, suitable examples being butylated hydroxyanisol (BHA) or butylated hydroxytoluene (BHT), which are used in conventional amounts, ordinarily from 0.01 to 0.5% [m/m], preferably 0.05 to 0.2% [m/m]. The active ingredient release can be controlled for example by adding so-called pore formers. These are for example sugars, especially lactose, polyols, especially mannitol or polyethylene glycols such as, for example, Macrogol 1500. The pore formers are employed in a concentration of from 5% to 40% [m/m], preferably in a concentration of from 5% to 20% [m/m]. Another possibility for influencing the active ingredient release is represented by addition of disintegration aids. It is possible to employ for this purpose so-called superdisintegrants such as crospovidone, croscarmellose sodium or crosslinked sodium carboxymethylstarch. The superdisintegrants are employed in a concentration of from 1% to 15% [m/m], preferably in a concentration of from 3% to 10% [m/m]. Substances which can be employed as alternative thereto are those which are soluble in acids and/or evolve carbon dioxide, such as magnesium carbonate or calcium carbonate. The carbon dioxide-releasing substances are employed in a concentration of from 5% to 15% [m/m], preferably in a concentration of from 5% to 10% [m/m].

It is possible to employ as pharmaceutically active substances active pharmaceutical ingredients, in particular those whose unpleasant taste is to be concealed.

Examples which may be mentioned are antibiotics such as, for example, quinolone antibiotics, this designation also being intended to include compounds derived from naphthyridone.

Quinolones, preferably fluoroquinolones, are inter alia compounds like those disclosed in the following documents: U.S. Pat. No. 4,670,444 (Bayer A G), U.S. Pat. No. 4,472,405 (Riker Labs), U.S. Pat. No. 4,730,000 (Abbott), U.S. Pat. No. 4,861,779 (Pfizer), U.S. Pat. No. 4,382,892 (Daiichi), U.S. Pat. No. 4,704,459 (Toyama); specific examples of quinolones which may be mentioned are pipemidic acid and nalidixic acid; examples of fluoroquinolones which may be mentioned are: benofloxacin, binfloxacin, cinoxacin, ciprofloxacin, danofloxacin, difloxacin, enoxacin, enrofloxacin, fleroxacin, ibafloxacin, levofloxacin, lomefloxacin, marbofloxacin, moxifloxacin, norfloxacin, ofloxacin, orbifloxacin, perfloxacin, temafloxacin, tosufloxacin, sarafloxacin, sparfloxacin.

A preferred group of fluoroquinolones are those of the formula (I) or (II):

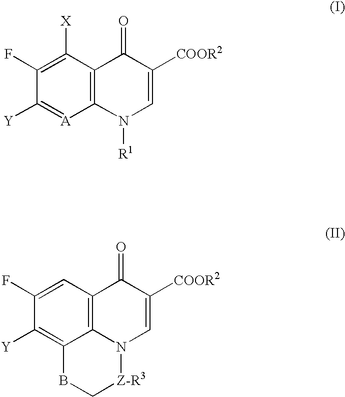

in which
X is hydrogen, halogen, C₁₋₄-alkyl, C₁₋₄-alkoxy, NH₂,
Y is radicals of the structures

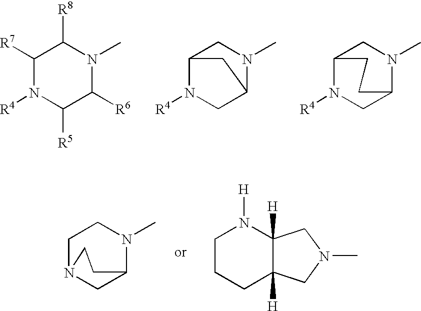

in which
R⁴ is optionally hydroxy- or methoxy-substituted straight-chain or branched C₁-C₄-alkyl, cyclopropyl, acyl having 1 to 3 C atoms,
R⁵ is hydrogen, methyl, phenyl, thienyl or pyridyl,
R⁶ is hydrogen or C₁₋₄-alkyl,
R⁷ is hydrogen or C₁₋₄-alkyl,
R⁸ is hydrogen or C₁₋₄-alkyl,
   and
R¹ is an alkyl radical having 1 to 3 carbon atoms, cyclopropyl, 2-fluoroethyl, methoxy, 4-fluorophenyl, 2,4-difluorophenyl or methylamino,
R² is hydrogen or optionally methoxy- or 2-methoxyethoxy-substituted alkyl having 1 to 6 carbon atoms, and cyclohexyl, benzyl, 2-oxopropyl, phenacyl, ethoxycarbonylmethyl, pivaloyloxymethyl,
R³ is hydrogen, methyl or ethyl, and
A is nitrogen, ═CH—, ═C(halogen)-, ═C(OCH₃)—, ═C(CH₃)— or ═C(CN),
B is oxygen, optionally methyl- or phenyl-substituted ═NH or ═CH₂,
Z is ═CH— or ═N—,
   and the pharmaceutically usable salts and hydrates thereof.

The compounds of the formulae (I) and (II) may be present in the form of their racemates or in enantiomeric forms.

Preference is given to compounds of the formula (I)

in which

### A is ═CH— or ═C—CN,

R¹ is optionally halogen-substituted C₁-C₃-alkyl or cyclopropyl,
R² is hydrogen or C₁₋₄-alkyl,
Y is radicals of the structures

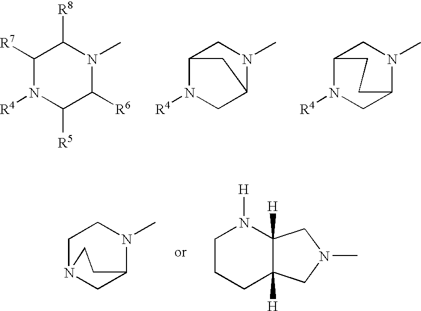

in which
R⁴ is optionally hydroxy-substituted straight-chain or branched C₁-C₃-alkyl, oxalkyl having 1 to 4 C atoms,
R⁵ is hydrogen, methyl or phenyl,
R⁷ is hydrogen or methyl,
R⁶ and R⁸ are hydrogen,
   and the pharmaceutically usable hydrates and salts thereof.

Particular preference is given to compounds of the formula (I)

in which

### A is ═CH— or ═C—CN,

R¹ is cyclopropyl,
R² is hydrogen, methyl or ethyl,
Y is radicals of the structures

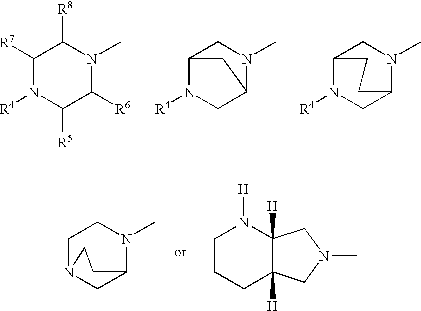

in which
R⁴ is methyl, optionally hydroxy-substituted ethyl,
R⁵ is hydrogen or methyl,
R⁷ is hydrogen or methyl,
R⁶ and R⁸ are hydrogen,
   and the pharmaceutically usable salts and hydrates thereof.

Suitable salts are pharmaceutically usable acid addition salts and basic salts.

Examples of pharmaceutically usable salts are the salts of hydrochloric acid, sulphuric acid, acetic acid, glycolic acid, lactic acid, succinic acid, citric acid, tartaric acid, methanesulphonic acid, 4-toluenesulphonic acid, galacturonic acid, gluconic acid, embonic acid, glutamic acid or aspartic acid. The compounds of the invention can also be bound to acidic or basic ion exchangers. Pharmaceutically usable basic salts which may be mentioned are the alkali metal salts, for example the sodium or potassium salts, the alkaline earth metal salts, for example the magnesium or calcium salts; the zinc salts, the silver salts and the guanidinium salts.

Hydrates mean both the hydrates of the fluoroquinolones themselves and the hydrates of the salts thereof.

Particularly preferred fluoroquinolones which may be mentioned are the compounds described in WO 97/31001, in particular 8-cyano-1-cyclopropyl-7-(1S,6S)-2,8-diazabicyclo[4.3.0]nonan-8-yl)-6-fluoro-1,4-dihydro-4-oxo-3-quinolinecarboxylic acid (pradofloxacin) having the formula

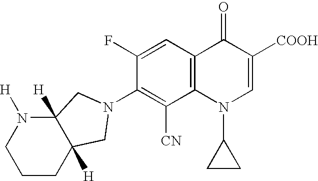

Pradofloxacin is preferably employed in its free form as anhydrate, e.g. in modification B (cf. WO 00/31076), or as trihydrate (cf. WO 2005/097 789).

Also particularly preferably employed is enrofloxacin:

### 1-Cyclopropyl-7-(4-ethyl-1-piperazinyl)-6-fluoro-1,4-dihydro-4-oxo-3-quinoline-carboxylic acid

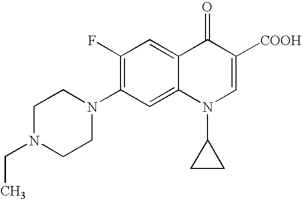

Besides enrofloxacin and pradofloxacin, mention may also be made as preferred quinolone anti-infectives of marbofloxacin, orbifloxacin, difloxacin and ibafloxacin.

Further suitable active pharmaceutical ingredients are for example triazinones such as, for example, diclazuril and in particular ponazuril and toltrazuril.

Mention may furthermore be made of 24-membered cyclic depsipeptides having an anthelmintic effect, e.g. PF 1022 and especially emodespide.

Other anthelmintics are also suitable. Examples which may be mentioned are epsiprantel and especially praziquantel.

Further active pharmaceutical ingredients which can be employed are pharmacologically acceptable phosphonic acid derivatives, these normally being organic compounds suitable as metabolic stimulants and tonics especially for productive and domestic animals. Preferred examples which may be mentioned are the compounds, which have been known for a long time, toldimfos and especially butaphosphan (e.g. used in the product Catosal®), which serve inter alia for mineral (phosphorus) supplementation.

Many other active pharmaceutical ingredients are also suitable in principle for use in the extrudates of the invention, because it is unnecessary to melt the active ingredient. Owing to the taste-masking effect of the extrudates, they are preferably suitable for active ingredients with an unpleasant—e.g. bitter-taste.

The incorporation in a lipophilic matrix allows—depending on the nature of the active ingredient employed—a delayed release and thus a slow-release effect also to be achieved.

It is possible for all pharmaceutically active ingredients—as explained above in detail for the quinolones—to use the corresponding pharmaceutically acceptable salts, hydrates, solvates and, where appropriate, different modifications.

Optically active substances can be used in the form of their stereoisomers or as stereoisomer mixture, e.g. as pure or enriched enantiomers or as racemates.

The amount of active ingredient employed in the extrudates depends on the potency and desired dosage. It emerges that extrudates with high active ingredient concentrations of up to 80% [m/m], preferably up to 70% [m/m], particularly preferably up to 60% [m/m] can also be produced. Normal concentration ranges are for example from 1 to 80% [m/m], preferably 5 to 70% [m/m], particularly preferably 30 to 60% [m/m].

The extrudates of the invention are produced by the starting materials (the pharmaceutically active substance(s), the base and, where appropriate, excipients and additives) being mixed and then extruded. The extrusions are preferably carried out at a temperature which does not lead to complete melting of the thermoformable materials and in particular normally at a temperature in the region of room temperature, preferably of 40° C., to below the melting range of the thermoformable materials. The extrusion process ought to be carried out with the material temperature as constant as possible. Suitable for this purpose are in particular heatable screw extruders, especially twin screw extruders. The extruded strand preferably has a round cross section and a diameter as indicated above. The extruded strand can be pelletized directly on extrusion with a knife or in a separate step by gentle grinding in a conventional mill, e.g. in a centrifugal mill. The particle size of the resulting product depends on the diameter of the die used, the maximum length of the pelletized strands corresponding to three times the strand diameter. Typical particle sizes are for example from 300 to 500 μm. In a preferred embodiment, the ground material can also be seived. The fines can be removed thereby.

The statement occasionally made herein that the extrudates are extruded below their melting point is to be understood to mean that the extrudates—as indicated above—are extruded at a temperature at which the employed thermoplastic base is not yet molten. Other ingredients such as, for example, the active ingredients often have a higher melting point.

With the extrudates, the active ingredient release is reduced when the strand diameter is smaller. Such extrudates are thus suitable for concealing the taste of ingredients with an unpleasant taste.

The extrudates of the invention can after gentle pelletization be processed further where appropriate to suitable pharmaceutical forms. Addition of further excipients is necessary where appropriate for the further processing. The pharmaceutical form which is preferred according to the invention is that of tablets which can where appropriate have shapes adapted to the desired use. Other suitable pharmaceutical forms are pastes, suspensions, sachets, capsules etc.

The extrudates and medicaments of the invention are generally suitable for use for humans and animals. They are preferably employed in animal management and animal breeding for productive and breeding livestock, zoo, laboratory, experimental and companion animals, especially for mammals.

The productive and breeding livestock include mammals such as, for example, cattle, horses, sheep, pigs, goats, camels, water buffalos, donkeys, rabbits, fallow deer, reindeer, fur-bearing animals such as, for example, mink, chinchilla, racoon, and birds such as, for example, chicken, geese, turkeys, ducks, pigeons and ostriches. Examples of preferred productive livestock are cattle, sheep, pigs and chickens.

The laboratory and experimental animals include dogs, cats, rabbits and rodents such as mice, rats, guinea pigs and golden hamsters.

Companion animals include dogs, cats, horses, rabbits, rodents such as golden hamsters, guinea pigs, mice, also reptiles, amphibia and birds for keeping at home and in zoos.

The extrudates are normally employed directly or in the form of suitable preparations (pharmaceutical forms) enterally, especially orally.

Enteral use of the active ingredients takes place for example orally in the form of granules, tablets, capsules, pastes, granulates, suspensions or medicated feed.

Suitable preparations are:

solid preparations such as, for example, granules, pellets, tablets, boli and active ingredients containing shaped articles.

Solid preparations are produced by mixing the active ingredients with suitable carriers, where appropriate with the addition of excipients, and converting into the desired form.

Carriers which may be mentioned are all physiologically tolerated solid inert materials. Inorganic and organic materials are used as such. Examples of inorganic materials are sodium chloride, carbonates such as calcium carbonate, bicarbonates, aluminium oxides, silicas, aluminas, precipitated or colloidal silicon dioxide, phosphates.

Examples of organic materials are sugars, cellulose, human and animal foodstuffs such as milk powder, animal meals, ground and crushed grains, starches.

Excipients are preservatives, antioxidants, colorants. Suitable excipients and the necessary amounts employed are known in principle to the skilled person. An example of a preservative which may be mentioned is sorbic acid. Examples of suitable antioxidants are butylated hydroxyanisole (BHA) or butylated hydroxytoluene (BHT). Suitable colorants are organic and inorganic colorants and pigments suitable for pharmaceutical purposes, such as, for example, iron oxide.

Further suitable excipients are lubricants and glidants such as, for example, magnesium stearate, stearic acid, talc, bentonites, disintegration promoting substances such as starch or crosslinked polyvinylpyrrolidone, binders such as, for example, starch, gelatin or linear polyvinylpyrrolidone, and dry binders such as microcrystalline cellulose.

Further adjuvants which can be employed are oils such as vegetable oils (e.g. olive oil, soya oil, sunflower oil) or oils of animal origin such as, for example, fish oil. Usual amounts are from 0.5 to 20% [m/m], preferably 0.5 to 10% [m/m], particularly preferably 1 to 2% [m/m].

Suspensions can be used orally. They are produced by suspending the active ingredient in a carrier liquid, where appropriate with the addition of further excipients such as wetting agents, colorants, absorption-promoting substances, preservatives, antioxidants, light stabilizers.

Suitable carrier liquids are homogeneous solvents or solvent mixtures in which the respective extrudates do not dissolve. Examples which may be mentioned are physiologically tolerated solvents such as water, alcohols such as ethanol, butanol, glycerol, propylene glycol, polyethylene glycols and mixtures thereof.

Wetting agents (dispersants) which can be employed are surfactants. Examples which may be mentioned are:

nonionic surfactants, e.g. polyoxyethylated castor oil, polyoxyethylated sorbitan monooleate, sorbitan monostearate, glycerol monostearate, polyoxyethyl stearate, alkylphenol polyglycol ethers;
ampholytic surfactants such as di-Na N-lauryl-β-iminodipropionate or lecithin;
anionic surfactants such as Na lauryl sulphate, fatty alcohol ether sulphates, mono/dialkyl polyglycol ether orthophosphoric ester monoethanolamine salt;
cationic surfactants such as cetyltrimethylammonium chloride.

Further excipients which may be mentioned are for example:

viscosity-increasing and suspension-stabilizing substances such as carboxymethyl-cellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silica or mixtures of the substances mentioned.

Semisolid preparations can be administered orally. They differ from the suspensions and emulsions described above only by their higher viscosity.

The active ingredients can also be employed in combination with synergists or with further active ingredients.

### EXAMPLES

Unless indicated otherwise, percentage date are percent by weight based on the finished mixture.

### I. Production of the Extrudates

A powder mixture consisting of the active ingredient enrofloxacin (50% [m/m]) and the excipients Compritol® 888 ATO (49% [m/m]), a fatty base with the main ingredient glycerol dibehenate (it also contains the mono- and triesters, and smaller amounts of esters with C₁₆-C₂₀ fatty acids), and Aerosil® 200 (1% [m/m]), a pyrogenic colloidal silicon dioxide whose use contributes to improving the flowability of the powder composition, is mixed before the extrusion in a laboratory mixer at room temperature (15 min, 40 rpm), and the powder mixture is transferred into the gravimetric feed unit of the extruder.

A co-rotating twin screw extruder with a round-section die and blunt screw attachments is used for the melt extrusion. The setting of the feed rate and the screw speed is adapted to the die plate used in order to ensure a reproducible process. The respective settings are listed in Tab. 1.

TABLE 1
Extrusion setting data

| Batch | Diameter [mm] | Screw speed [rpm] | Feed rate [g/min] |
|---|---|---|---|
| 1 | 0.3 | 18 | 30 |
| 2 | 0.4 | 20 | 30 |
| 3 | 0.5 | 20 | 30 |
| 4 | 1.0 | 30 | 40 |
| 5 | 2.7 | 30 | 50 |
| 6 | 5.0 | 30 | 50 |

6 different die plates are used to produce the different batches. They differ in their die diameter, number of dies and die lengths. Care is taken in this connection that for die plates having die diameters less than or equal to 1.0 mm the open area and the ratio of length to diameter of the dies are kept constant in order to be able to assume that the stress on the extrudate composition is always the same. Tab. 2 shows the respective parameters of the individual die plates.

TABLE 2
Die plate parameters

| Diameter of the dies [mm] | 5.0 | 2.7 | 1.0 | 0.5 | 0.4 | 0.3 |
|---|---|---|---|---|---|---|
| Number of dies | 1 | 3 | 3 | 12 | 19 | 33 |
| Length of the dies [mm] | 5.0 | 7.5 | 2.5 | 1.25 | 1.0 | 0.75 |
| Ratio of length to diameter | 1 | 2.8 | 2.5 | 2.5 | 2.5 | 2.5 |
| Open area [mm²] | 19.64 | 17.18 | 2.36 | 2.36 | 2.39 | 2.33 |

The melt extrusions always take place at the same temperatures and are carried out below the melting range of Compritol® 888 ATO (approx. 70° C.). The temperature at the die plate was 60° C., and the temperatures of the barrels of the extruder from the die plate in the direction of the powder feed were as follows: 60° C., 55° C., 55° C., 55° C., 55° C., 25° C., 25° C., 25° C. After the melt extrusion, the extrudates were ground with a centrifugal mill at 6000 rpm, a 12-tooth rotor and a sieve insert with 1.5 mm conidur perforations. The 315-400 μm sieve fraction of each batch is used for all the investigations.

Further formulations for extrudates (unless indicated otherwise, the percentage data are % by weight):

### Example 2

| | |
|---|---|
| Praziquantel | 50% |
| Glyceryl behenate (Compritol ® 888 ATO) | 49% |
| Colloidal silicon dioxide (Aerosil ® 200) | 1% |

The three starting materials are mixed and extruded (die diameter: 0.4 mm, temperature of the die plate 60° C. Further processing of the extruded strands can take place as in Example 1.

### Example 3

| | |
|---|---|
| Emodepside | 50% |
| Glyceryl behenate (Compritol ® 888 ATO) | 49% |
| Colloidal silicon dioxide (Aerosil ® 200) | 1% |

The three starting materials are mixed and extruded (die diameter: 0.5 mm, temperature of the die plate 60° C.). Further processing of the extruded strands can take place as in Example 1.

### Example 4

| | |
|---|---|
| Praziquantel | 50% |
| Glycerol trimyristate (Dynasan 114 ®) | 49% |
| Colloidal silicon dioxide (Aerosil ® 200) | 1% |

The three starting materials are mixed and extruded (die diameter: 0.5 mm, temperature of the die plate 50° C.). Further processing of the extruded strands can take place as in Example 1.

### Example 5

| | |
|---|---|
| Emodepside | 50% |
| Glycerol trimyristate (Dynasan 114 ®) | 49% |
| Colloidal silicon dioxide (Aerosil ® 200) | 1% |

The three starting materials are mixed and extruded (die diameter: 0.4 mm, temperature of the die plate 50° C.). Further processing of the extruded strands can take place as in Example 1.

### Example 6

| | |
|---|---|
| Praziquantel | 50% |
| Glycerol trimyristate (Dynasan 114 ®) | 49% |
| Colloidal silicon dioxide (Aerosil ® 200) | 1% |

The three starting materials are mixed and extruded (die diameter: 0.33 mm, temperature of the die plate 50° C.). Further processing of the extruded strands can take place as in Example 1.

### Example 7

| | |
|---|---|
| Praziquantel | 50% |
| Glycerol tripalmitate (Dynasan 116 ®) | 49% |
| Colloidal silicon dioxide (Aerosil ® 200) | 1% |

The three starting materials are mixed and extruded (die diameter: 0.33 mm, temperature of the die plate 56° C.). Further processing of the extruded strands can take place as in Example 1.

### Example 8

| | |
|---|---|
| Praziquantel | 50% |
| Glycerol tristearate (Dynasan 118 ®) | 49% |
| Colloidal silicon dioxide (Aerosil ® 200) | 1% |

The three starting materials are mixed and extruded (die diameter: 0.33 mm, temperature of the die plate 65° C.). Further processing of the extruded strands can take place as in Example 1.

### Example 9

| | |
|---|---|
| Praziquantel | 50% |
| Glycerol trimyristate (Dynasan 114 ®) | 49% |
| Colloidal silicon dioxide (Aerosil ® 200) | 1% |

The three starting materials are mixed and extruded (die diameter: 0.4 mm, temperature of the die plate 50° C.). Further processing of the extruded strands can take place as in Example 1.

### Example 10

| | |
|---|---|
| Praziquantel | 50% |
| Glycerol tripalmitate (Dynasan 116 ®) | 49% |
| Colloidal silicon dioxide (Aerosil ® 200) | 1% |

The three starting materials are mixed and extruded (die diameter: 0.4 mm, temperature of the die plate 56° C.). Further processing of the extruded strands can take place as in Example 1.

### Example 11

| | |
|---|---|
| Praziquantel | 50% |
| Glycerol tristearate (Dynasan 118 ®) | 49% |
| Colloidal silicon dioxide (Aerosil ® 200) | 1% |

The three starting materials are mixed and extruded (die diameter: 0.4 mm, temperature of the die plate 65° C.). Further processing of the extruded strands can take place as in Example 1.

### Example 12

| | |
|---|---|
| Emodepside | 50% |
| Glycerol tripalmitate (Dynasan 116 ®) | 49% |
| Colloidal silicon dioxide (Aerosil ® 200) | 1% |

The three starting materials are mixed and extruded (die diameter: 0.33 mm, temperature of the die plate 56° C.). Further processing of the extruded strands can take place as in Example 1.

### Example 13

| | |
|---|---|
| Emodepside | 50% |
| Glycerol tristearate (Dynasan 118 ®) | 49% |
| Colloidal silicon dioxide (Aerosil ® 200) | 1% |

The three starting materials are mixed and extruded (die diameter: 0.33 mm, temperature of the die plate 65° C.). Further processing of the extruded strands can take place as in Example 1.

### Example 14

| | |
|---|---|
| Emodepside | 50% |
| Glycerol trimyristate (Dynasan 114 ®) | 49% |
| Colloidal silicon dioxide (Aerosil ® 200) | 1% |

The three starting materials are mixed and extruded (die diameter: 0.33 mm, temperature of the die plate 50° C.). Further processing of the extruded strands can take place as in Example 1.

### Example 15

| | |
|---|---|
| Emodepside | 50% |
| Glycerol tripalmitate (Dynasan 116 ®) | 49% |
| Colloidal silicon dioxide (Aerosil ® 200) | 1% |

The three starting materials are mixed and extruded (die diameter: 0.4 mm, temperature of the die plate 56° C.). Further processing of the extruded strands can take place as in Example 1.

### Example 16

| | |
|---|---|
| Emodepside | 50% |
| Glycerol tristearate (Dynasan 118 ®) | 49% |
| Colloidal silicon dioxide (Aerosil ® 200) | 1% |

The three starting materials are mixed and extruded (die diameter: 0.4 mm, temperature of the die plate 65° C.). Further processing of the extruded strands can take place as in Example 1.

### Example 17

| | |
|---|---|
| Butafosfan | 50% |
| Butylated hydroxytoluene | 0.1% |
| Glycerol trimyristate (Dynasan 114 ®) | 48.9% |
| Colloidal silicon dioxide (Aerosil ® 200) | 1% |

The starting materials are mixed and extruded (die diameter: 0.4 mm, temperature of the die plate 50° C.). Further processing of the extruded strands can take place as in Example 1.

### Example 18

| | |
|---|---|
| Praziquantel | 50% |
| Glycerol trimyristate (Dynasan 114 ®) | 39% |
| Polyethylene glycol 1500 | 10% |
| Colloidal silicon dioxide (Aerosil ® 200) | 1% |

The starting materials are mixed and extruded (die diameter: 0.33 mm, temperature of the die plate 50° C.). Further processing of the extruded strands can take place as in Example 1.

### Example 19

| | |
|---|---|
| Emodepside | 50% |
| Glycerol trimyristate (Dynasan 114 ®) | 39% |
| Polyethylene glycol 1500 | 10% |
| Colloidal silicon dioxide (Aerosil ® 200) | 1% |

The starting materials are mixed and extruded (die diameter: 0.33 mm, temperature of the die plate 50° C.). Further processing of the extruded strands can take place as in Example 1.

### Example 20

| | |
|---|---|
| Butafosfan | 50% |
| Butylated hydroxytoluene | 0.1% |
| Glycerol trimyristate (Dynasan 114 ®) | 38.9% |
| Polyethylene glycol 1500 | 10% |
| Colloidal silicon dioxide (Aerosil ® 200) | 1% |

The starting materials are mixed and extruded (die diameter: 0.33 mm, temperature of the die plate 50° C.). Further processing of the extruded strands can take place as in Example 1.

### II. Long-Term Investigation of Medicinal Substance Release

Long-term investigations on the medicinal substance release are carried out using the release system according to Ph. Eur. 2.9.3, Apparatus 2. Also used is a sinker vessel in which the sample is located. The sinker vessel lies on the bottom of the release vessel, and the distance from the lower edge of the paddle stirrer is 2.5 cm. All the measurements are carried out with a paddle stirrer at 50 rpm in 900 ml of medium at 37° C.±0.5° C. for 6 samples per batch. The release is carried out at a pH of 7.4 (according to USP27 “Buffer Solutions”) with the addition of 0.001% Polysorbate 20.

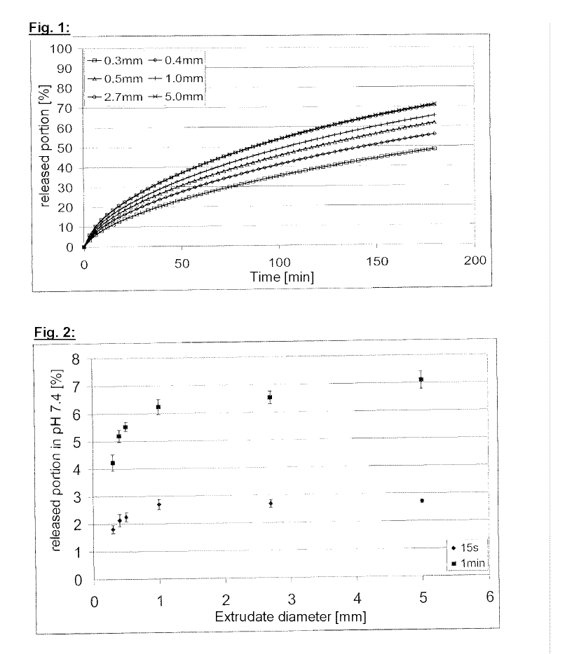

A difference emerges in the release profiles in the medium of pH 7.4, which corresponds to the pH range in the mouth (see FIG. 1: Release profiles in pH 7.4 of all the investigated batches [means from 6 determinations]). It is unambiguously evident here that enrofloxacin release per unit time increases as the original diameter of the extrudates increases. There is no difference in the release profile for ground products from extrudates of the two largest original diameters.

### III. Short-Term Investigation of the Medicinal Substance Release

It is not possible for technical reasons to carry out short-term investigations of the initial release with the method for the long-term release investigations. The following method is therefore used for short-term investigations:

A disintegration tester with 700 ml of medium of pH 7.4 (as in the long-term investigations) at 37° C.±0.5° C. is used for these investigations. The samples are distributed in three sinker vessels, these are introduced into the sample holder (according to Ph. Eur. 5.5, 2.9.1. Apparatus for Test B) and the test is carried out for 15 s or 1 min The rate of raising and lowering the sample holder is constant in all the tests. For comparison with the long-term investigations, 60 min tests according to the short-term test scheme are also carried out.

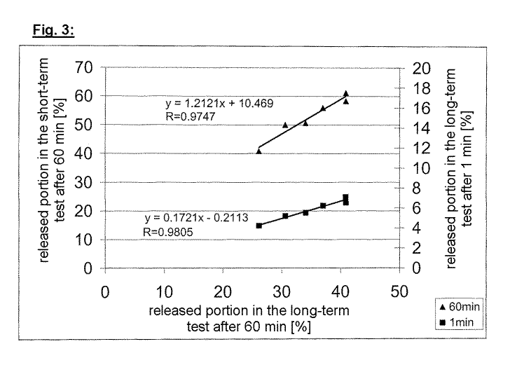

FIG. 2 shows the results from the short-term tests after 15 s and 1 min (mean±standard deviations from 6 samples). The released amount of active ingredient is plotted against the strand diameter of the batches. It is quite clear that the active ingredient released per unit time decreases as the strand diameter decreases. A distinct decrease in release is to be observed especially for strand diameters below 0.5 mm.

To confirm the possibility of comparing the two release investigations employed, the results of the long-term investigation are correlated with those of the short-term investigation. The 1 min and 60 min test values from the short-term test are in each case associated with the data of the long-term study. It is very easily possible to correlate the values; there is a linear relationship (see FIG. 3). Each point in the diagram corresponds to a particular diameter.
Adeyeye C. M., Price J. C., Development and Evaluation of Sustained-Release Ibuprofen-Wax Microspheres 0.1. Effect of Formulation Variables on Physical Characteristics, Pharmaceutical Research, 1991; (8): 1377-1383.
Adeyeye C. M., Price J. C., Development and Evaluation of Sustained-Release Ibuprofen-Wax Microspheres 0.2. In-Vitro Dissolution, Pharmaceutical Research, 1994; (11): 575-579.
Al-Omran M. F., Al-Suwayeh S. A., El-Helw A. M., Saleh S. I, Taste masking of diclofenac sodium using microencapsulation, Journal of Microencapsulation, 2002; (19): 45-52.
Barra J., Lescure F., Doelker E., Taste masking as a consequence of the organisation of powder mixes, Pharmaceutica Acta Helvetiae, 1999; (74): 37-42.
Barthelemy P., Laforet J. P., Farah N., Joachim J., Compritol (R) 888 ATO: an innovative hot-melt coating agent for prolonged-release drug formulations, European Journal of Pharmaceutics and Biopharmaceutics, 1999; (47): 87-90.
Bienz M., Taste masking strategies for drug dosage forms, Manufacturing Chemist, 1996; (67): 17-20.
Breitkreutz J., El-Saleh F., Kiera C., Kleinebudde P., Wiedey W., Pediatric drug formulations of sodium benzoate: II. Coated granules with a lipophilic binder, European Journal of Pharmaceutics and Biopharmaceutics, 2003; (56): 255-260.
Cerea M., Zheng W. J., Young C. R., McGinity J. W., A novel powder coating process for attaining taste masking and moisture protective films applied to tablets, International Journal of Pharmaceutics, 2004; (279): 127-139.
Chun M K., Choi H. K., Preparation and characterization of enrofloxacin/carbopol complex in aqueous solution, Archives of Pharmacal Research, 2004; (27): 670-675.
Katsuragi Y., Mitsui Y., Umeda T., Otsuji K., Yamasawa S., Kurihara K., Basic studies for the practical use of bitterness inhibitors: Selective inhibition of bitterness by phospholipids, Pharmaceutical Research, 1997; (14): 720-724.
Kim E.-H., Choi H. K., Preparation of various solid-lipid beads for Drug Delivery of Enrofloxacin, Drug Delivery, 2004; (11): 365-370.
Li F.-Q., Hu Deng J.-X., Su H., Xu S., Liu J.-Y., In vitro controlled release of sodium ferulate from Compritol 888 ATO-based matrix tablets, International Journal of Pharmaceutics, 2006; (324): 152-157.
Li S. P., Marteliucci S. A., Bruce R. D., Kinyon A. C., Hay M. B., Higgins J. D., Evaluation of the film-coating properties of a hydroxyethyl cellulose/hydroxypropyl methylcellulose polymer system, Drug Development and Industrial Pharmacy, 2002; (28): 389-401.
Lovrecich M., Nobile F., Rubessa F., Zingone G., Effect of ageing on the release of indomethacin from solid dispersions with Eudragits, International Journal of Pharmaceutics, 1996; (131): 247-255.
Lu M. Y. F., Borodkin S., Woodward L., Li P., Diesner C., Hernandez L., Vadnere M., A Polymer Carrier System for Taste Masking of Macrolide Antibiotics, Pharmaceutical Research, 1991; (8): 706-712.
Mirghani A., Idkaidek N. M., Salem M. S., Najib N. M., Formulation and release behavior of diclofenac sodium in Compritol 888 matrix beads encapsulated in alginate, Drug Development and Industrial Pharmacy, 2000; (26): 791-795.
Ohta M., Buckton G., The use of inverse gas chromatography to assess the acid-base contributions to surface energies of cefditoren pivoxil and methacrylate copolymers and possible links to instability, International Journal of Pharmaceutics, 2004; (272): 121-128.
Pearnchob N., Dashevsky A., Siepmann J., Bodmeier R., Shellac used as coating material for solid pharmaceutical dosage forms: understanding the effects of formulation and processing variables, Stp Pharma Sciences, 2003a; (13): 387-396.
Pearnchob N., Siepmann J., Bodmeier R., Pharmaceutical applications of shellac: Moisture-protective and taste-masking coatings and extended-release matrix tablets, Drug Development and Industrial Pharmacy, 2003b; (29): 925-938.
Petereit H. U., Weisbrod W., Formulation and process considerations affecting the stability of solid dosage forms formulated with methacrylate copolymers, European Journal of Pharmaceutics and Biopharmaceutics, 1999; (47): 15-25.
Prompruk K., Govender T., Zhang S., Xiong C. D., Stolnik S., Synthesis of a novel PEG-block-poly(aspartic acid-stat-phenylalanine) copolymer shows potential for formation of a micellar drug carrier, International Journal of Pharmaceutics, 2005; (297): 242-253.
Reitz C, Kleinebudde P., Solid lipid extrudion of sustained release dosage forms. European Journal Of Pharmaceutics and Biopharmaceutics, 2007 doi:10.1016/j.ejpb.2007.03,008
Roy G., Taste masking in oral pharmaceuticals, Pharmaceutical technology Europe, 1994; (18): 84-99.
Schubert M. A. S. B. C., Thermal analysis of the crystallization and melting behavior of lipid matrices and lipid nanoparticles containing high amounts of lecithin, International Journal of Pharmaceutics, 2005; (298): 242-254.
Shirai Y., Sogo K., Yamamoto K., Kojima K., Fujioka H., Makita H., Nakamura Y., A Novel Fine Granule System for Masking Bitter Taste, Biological & Pharmaceutical Bulletin, 1993; (16): 172-177.
Sohi H., Sultana Y., Khar R. K., Taste masking technologies in oral pharmaceuticals: Recent developments and approaches, Drug Development and Industrial Pharmacy, 2004; (30): 429-448.
Suzuki H., Onishi H., Hisamatsu S., Masuda K., Takahashi Y., Iwata M., Machida Y., Acetaminophen-containing chewable tablets with suppressed bitterness and improved oral feeling, International Journal of Pharmaceutics, 2004; (278): 51-61.
Suzuki H., Onishi H., Takahashi Y., Iwata M., Machida Y., Development of oral acetaminophen chewable tablets with inhibited bitter taste, International Journal of Pharmaceutics, 2003; (251): 123-132.
Takagi S., Toko K., Wada K., Ohki T., Quantification of suppression of bitterness using an electronic tongue, Journal of Pharmaceutical Sciences, 2001; (90): 2042-2048.
Zhou F., Vervaet C., Remon J. P., Matrix pellets based on the combination of waxes, starches and maltodextrins, International Journal of Pharmaceutics, 1996; (133): 155-160.

Zhou F., Vervaet C., Schelkens M., Lefebvre R., Remon J. P., Bioavailability of ibuprofen from matrix pellets based on the combination of waxes and starch derivatives, International Journal of Pharmaceutics, 1998; (168): 79-84.

1.-10. (canceled)

11. A process for making a medicinal extrudate having a strand diameter of 0.5 mm or less, the process comprising:
a. mixing a pharmaceutically active substance and a thermoformable material to form a mixture;
b. extruding the mixture, at a temperature such that the thermoplastic base is not yet molten, to form an extrudate having a strand diameter of 0.5 mm or less.

12. The process of claim 11, wherein the pharmaceutically active substance is selected from the group consisting of enrofloxacin, pradofloxacin, marbofloxacin, orbifloxacin, difloxacin, ibafloxacin, diclazuril, ponazuril, toltrazuril, emodepside, epsiprantel, praziquantel, toldimfos, butaphosphan, and combinations thereof.

13. The process of claim 11, wherein the pharmaceutically active substance comprises 5 to 70% by weight of the extrudate.

14. The process of claim 11, wherein the thermoformable material is selected from the group consisting of polymers, lipids, surfactants, macrogols, sugars, sugar alcohols, and combinations thereof.

15. The process of claim 11, wherein the thermoformable material is a lipid.

16. The process of claim 15, wherein the lipid is a glycerol ester selected from the group consisting of glyceryl behenate, glycerol trimyristate, glycerol tripalmitate, glycerol tristearate, and combinations thereof.

17. The process of claim 11, wherein the thermoformable material comprises 25 to 80% by weight of the extrudate.

18. The process of claim 11, further comprising mixing an excipient into the mixture.

19. The process of claim 18, wherein the excipient is selected from the group consisting of a flow regulator, lubricant, surfactant, antioxidant, pore former, disintegration aid, and combinations thereof.

20. The process of claim 19, wherein the excipient is a flow regulator and wherein the flow regulator is colloidal silicon dioxide.

21. A pharmaceutical medicament prepared by the process of claim 11.

22. A process for making a medicament, the process comprising:
c. mixing a pharmaceutically active substance and a thermoformable material to form a mixture;
d. extruding the mixture, at a temperature such that the thermoplastic base is not yet molten, to form an extrudate having a strand diameter of 0.5 mm or less;
e. pelletizing the extrudate and forming a medicament.

23. The process of claim 22, further comprising mixing an excipient into the mixture.

24. The process of claim 23, wherein the excipient is selected from the group consisting of a flow regulator, lubricant, surfactant, antioxidant, pore former, disintegration aid, and combinations thereof.

25. The process of claim 24, wherein the excipient is a flow regulator and wherein the flow regulator is colloidal silicon dioxide.

26. A pharmaceutical medicament prepared by the process of claim 22.
